(19) United States
(12) Patent Application Publication
(10) Pub. No.: US 2025/0093290 A1
(43) Pub. Date: Mar. 20, 2025

(54) ELECTROCHEMICAL ARTIFICIAL MUSCLE SYSTEM AND ELECTROCHEMICAL ARTIFICIAL MUSCLE TESTING DEVICE

(71) Applicant: SUZHOU INSTITUTE OF NANO-TECH AND NANO-BIONICS (SINANO) , CHINESE ACADEMY OF SCIENCES, Suzhou (CN)

(72) Inventors: Jiangtao DI, Suzhou (CN); Ming REN, Suzhou (CN); Panpan XU, Suzhou (CN); Qingwen LI, Suzhou (CN)

(73) Assignee: SUZHOU INSTITUTE OF NANO-TECH AND NANO-BIONICS (SINANO) , CHINESE ACADEMY OF SCIENCES, Suzhou (CN)

(21) Appl. No.: 18/290,809

(22) PCT Filed: Dec. 9, 2022
(86) PCT No.: PCT/CN2022/137918

§ 371 Date: Jan. 22, 2024

(30) Foreign Application Priority Data
Mar. 15, 2022 (CN) 202210256161.1

(51) Int. Cl. G01N 27/30

(52) U.S. Cl. CPC G01N 27/30

(57) ABSTRACT

An electrochemical artificial muscle system and an electrochemical artificial muscle testing device are provided. The electrochemical artificial muscle system includes a working electrode including a conductive yarn having a helical structure; a counter electrode containing selected metal elements; an electrolyte including an ionic liquid containing selected ions, the selected ions including the selected metal elements; where the working electrode and the counter electrode are all in contact with the electrolyte. The artificial muscle system based on an aluminum ion battery system has a contractile retention (catch state) characteristic, i.e., artificial muscle yarns contract when a voltage is applied, and after the voltage is removed, the contraction state of the artificial muscle yarn is remained, within almost no any decay within 450 s. The artificial muscle yarn has good energy storage properties with a capacity of 20-100 mAh·g⁻¹ so that the energy for injecting artificial muscle during the driving is stored.

### CROSS-REFERENCE TO THE RELATED APPLICATIONS

This application is the national phase entry of International Application No. PCT/CN2022/137918, filed on Dec. 9, 2022, which is based upon and claims priority to Chinese Patent Application No. 202210256161.1, filed on Mar. 15, 2022, the entire contents of which are incorporated herein by reference.

### TECHNICAL FIELD

The present application particularly relates to an electrochemical artificial muscle system and an electrochemical artificial muscle testing device, belonging to the technical field of material science.

### BACKGROUND

The artificial muscle yarn, as a class of smart material system, can generate reversible volume deformation under external stimuli generally including electrical stimuli, light stimuli, humidity/solvent stimuli, electrochemical stimuli, etc. Due to the large contractile stroke, large contractile stress, and high power/energy density, artificial muscle yarn has exhibited wide application prospects in the fields of soft robots, mechanical exoskeletons, etc. There are a series of materials being studied, for example, carbon nanotube yarns, shape memory alloy wires, graphene fibers, nylon polymer fibers, liquid crystal elastomer fibers, etc. Artificial muscles based on shape memory alloy wires can achieve 16° mm⁻¹ rotation under the stimulus of Joule heating, with a maximum rotation speed of 10500 rpm.

At present, an artificial muscle fiber based on a liquid crystal elastomer has been reported in Science Robotics. The liquid crystal elastomer fiber was prepared by an electrospinning technology. This fiber can generate 60% contractile stroke under the stimulus of near-infrared light, and the response speed can reach 120% s⁻¹. Prof. Peng Huisheng's research team reported a solvent-driven artificial muscle yarn based on multi-level carbon nanotube fibers. Under the action of ethanol vapor, the fiber can contract by 60%. At the same time, due to the presence of multi-level pores, the fiber can quickly respond. The flexible fabric prepared based on this fiber can increase its weight by 100 times copper balls.

The energy density generated by the electrochemical artificial muscle fiber with a core-shell structure based on carbon nanotube fibers is as high as 2.35 J g⁻¹. However, among the artificial muscles reported at present, the actuation performance of almost all of the artificial muscles can be kept only under the condition that external stimuli always exist. When the external stimuli are removed, the contractile stroke of the fiber also disappears, which requires continuous external stimuli during the contractile process, so that the actuation process consumes much energy. In addition, during the actuation, only a small portion of energy is converted into mechanical energy, while the remaining energy is dissipated.

### SUMMARY

The main objective of the present application is to provide an electrochemical artificial muscle system and an electrochemical artificial muscle testing device to overcome the defects in the prior art.

To achieve the objective of the foregoing application, the technical solution adopted by the present application is as follows:

The embodiments of the present application provide an electrochemical artificial muscle system, comprising:
a working electrode comprising a conductive fiber having a coiled or helical structure;
a counter electrode containing selected metal elements;
an electrolyte comprising an ionic liquid containing selected ions, the selected ions comprising the selected metal elements;
wherein, the working electrode and the counter electrode are all in contact with the electrolyte, when a specified voltage is applied to the working and counter electrodes to charge the electrochemical artificial muscle system, the selected ions can be embedded into the conductive fiber so that the conductive fiber is contractile along the self-length direction, and when the specified voltage applied to the working and counter electrodes is removed, the inserted ions are lock in the conductive yarn and make the conductive yarn keep a contraction state;
when the electrochemical artificial muscle system is externally discharged, the selected ions can be de-inserted from the conductive yarn, so that the conductive yarn is restored to its initial length.

The embodiments of the present application further provide an electrochemical artificial muscle testing device, comprising:
the electrochemical artificial muscle system, one end of the working electrode being fixed; and,
a load fixedly connected with the other end of the working electrode and capable of pulling the working electrode along the length direction of the working electrode; and
a displacement monitoring mechanism at least used for measuring the displacement of the load.

Compared with the prior art, the present application has the following advantages:
1) the artificial muscle system based on an aluminum ion battery system provided by the embodiment of the present invention has a contraction retention characteristic (catch state), i.e., artificial muscle yarns contact when the voltage is applied, and after the voltage is removed, the contraction state of the artificial muscle yarns is remained, within almost no any decay within 450 s;
2) the artificial muscle system based on an aluminum ion battery system provided by the embodiment of the present invention couples an actuation function with energy storage to store external input energy during the actuation of the artificial muscle fibers, and the stored energy can power electrical appliances, thereby avoiding the waste of the energy;
3) the artificial muscle system based on an aluminum ion battery system provided by the embodiment of the present invention is simple in the fabricating process, can be produced on a large scale, and is beneficial to productization.

### BRIEF DESCRIPTION OF THE DRAWINGS

FIG. 1 is a structural diagram of an electrochemical artificial muscle testing device provided in example 1 of the present application;

FIG. 2 shows a contractile stroke curve of an artificial muscle fiber provided in example 1 of the present application;

FIG. 3 shows the contractile stroke of a carbon nanotube yarn after a square-wave voltage of 2.2 V is applied;

FIG. 4 shows a galvanostatic charge/discharge curve of a two-electrode aluminum ion battery system provided in example 1 under different current densities.

### DETAILED DESCRIPTION OF THE EMBODIMENTS

Because of the defects in the prior art, the inventor of this case, via long-term research and lots of practice, proposes the technical solution of the present application. Next, the technical solution, implementation process, and principle will be further explained and illustrated.

The embodiment of the present application provides an electrochemical artificial muscle system based on a battery system. The electrochemical artificial muscle system can generate reversable volume expansion when selected ions are insertion and de-insertion, and then the contraction and relaxation are achieved macroscopically. Furthermore, the contraction state of the electrochemical artificial muscle system can be kept after the removal of external stimuli (applied voltage).

Meanwhile, the embodiment of the present application provides an electrochemical artificial muscle system based on a battery system. The electrochemical artificial muscle system couples actuation with energy storage function so that the electrochemical artificial muscle system stores electrochemical energy while being driven, thereby providing energy for other electrical appliances.

The embodiment of the present application provides an electrochemical artificial muscle system, comprising:
a working electrode comprising a conductive yarn having a coiled or helical structure;
a counter electrode containing selected metal elements;
an electrolyte comprising an ionic liquid containing selected ions, the selected ions comprising the selected metal elements;
wherein, the working electrode and the counter electrode are all in contact with the electrolyte, when a specified voltage is applied to the working and counter electrodes to charge the electrochemical artificial muscle system, the selected ions can be inserted into the conductive yarn so that the conductive yarn contracts along the self-length direction, and when the specified voltage applied to the working and counter electrodes is removed, the inserted ions are lock in the conductive yarn and make the conductive yarn keep a contraction state;
when the electrochemical artificial muscle system is externally discharged, the selected ions can detach from the conductive yarn, so that the conductive fiber is restored to an initial length.

In a specific embodiment, the working electrode is obtained by twisting the conductive yarn until it forms a coiled structure.

In a specific embodiment, the twist density of the conductive yarn with the coiled structure is 1000 r/m-5000 r/m.

In a specific embodiment, the diameter of the conductive yarn with the coiled structure is 10 μm-500 μm.

In a specific embodiment, the conductive yarn comprises one or a combination of more than two of a carbon nanotube yarn, a graphene yarn, a graphite fiber, and a carbon fiber, but is not limited thereto.

In a specific embodiment, the selected ion comprises any one of AlCl₄⁻, a lithium-ion, and a zinc ion, but is not limited thereto.

In a specific embodiment, the ionic liquid comprises an EMImAlCl₄ ionic liquid or a salt solution containing lithium ions and/or zinc ions but is not limited thereto.

In a specific embodiment, the metal element contained in the counter electrode comprises any one or more than two aluminum, lithium, and zinc elements, but is not limited thereto.

In a specific embodiment, the material of the counter electrode comprises an alloy formed by any one or more than two metals selected from aluminum, lithium, and zinc, but is not limited thereto.

In a specific embodiment, when the specified voltage applied to the working and counter electrodes is removed, the longest time when the conductive fiber keeps the contraction state is 450 s.

In a specific embodiment, the specified voltage is 0-2.2 V.

In a specific embodiment, the electrochemical capacity of the working electrode is 20-100 mAh·g⁻¹, preferably 44 mAh·g⁻¹, and energy injected into the electrochemical artificial muscle system can be stored to reduce energy loss.

The embodiment of the present application further provides an electrochemical artificial muscle testing device, comprising:
the electrochemical artificial muscle system, one end of the working electrode being fixed on an electrode clamp; and,
a load fixedly connected with the other end of the working electrode and capable of pulling the working electrode along the length direction of the working electrode; and
a displacement monitoring mechanism at least used for measuring the displacement of the load.

In a specific embodiment, the displacement monitoring mechanism comprises a displacement sensor.

In a specific embodiment, the electrochemical artificial muscle testing device further comprises a cathode and an anode, the cathode being electrically connected with the working electrode, the anode being electrically connected with the counter electrode, the cathode and the anode being used for being connected with a power source and charging the electrochemical artificial muscle system, and for being electrically connected with the load and discharging the electrochemical artificial muscle system.

Next, the technical solution, implementation process, and principle will be further explained and illustrated in combination with drawings and specific embodiments. Unless otherwise stated, the conductive yarn, electrodes, electrolyte, displacement sensor, load, power source, and the like used in the embodiments of the present application can be commercially available, and their specific structure and parameters will not be defined and illustrated herein.

### Example 1

A fabricating method of an electrochemical artificial muscle testing device can comprise the following processes:

### 1) Preparation of Artificial Muscle Fibers

First, carbon nanotube yarns were twisted. The specific operation steps were as follows: one end of the carbon nanotube fiber hung vertically on a twisting motor, and the other end of the carbon nanotube fiber hung with a certain weight (the mass of the weight was 1-10 g), to twist the carbon nanotube fiber at the rotation speed of 0-200 rpm until a helical structure was formed;

### 2) Construction of Testing System

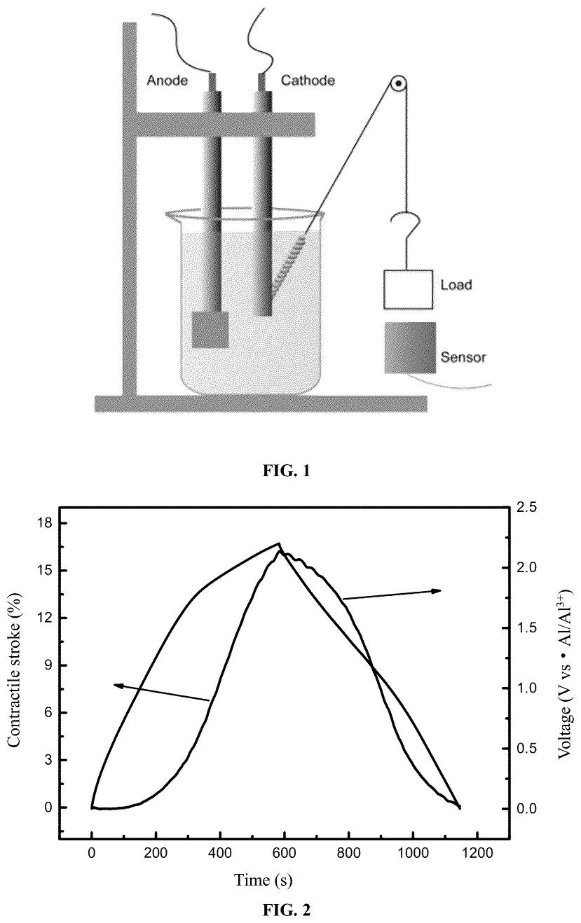

Anhydrous aluminum chloride was added into [EMIm]Cl to obtain an EMImAlCl₄ ionic liquid, and the EMImAlCl₄ ionic liquid was used as an electrolyte;
the carbon nanotube yarn and the aluminum foil were each independently in contact with the electrolyte by using the twisted carbon nanotube yarn as a working electrode and an aluminum foil as a counter electrode, to construct a two-electrode aluminum ion battery system;
one end of the working electrode was electrically connected to a cathode and the other end of the working electrode to a load, a connecting wire connecting the working electrode with the load bypassed a pulley to pull the load along the length direction of the working electrode; the counter electrode was electrically connected with the anode;
the displacement amount of the load was monitored by using the displacement sensor, and the structure of the formed testing system is shown in FIG. 1;

### 3) Testing Characterization

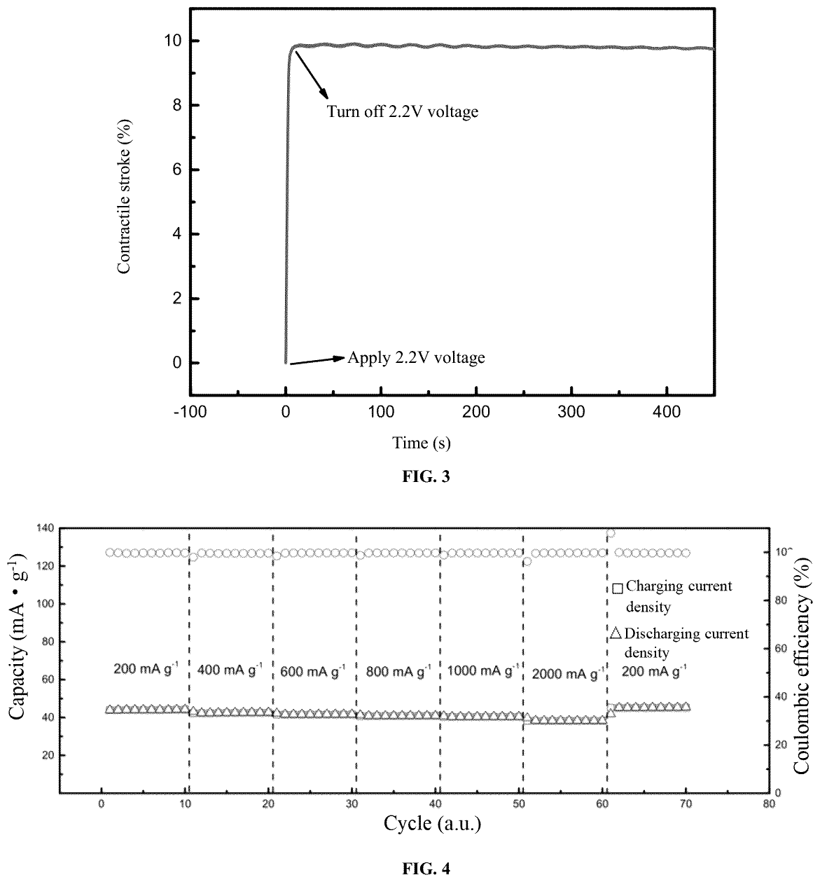

At the current density of 0.2 A g⁻¹, the two-electrode aluminum ion battery system was charged and discharged under the constant current, and the contractile stroke of the carbon nanotube yarn serving as the working electrode was measured by using the displacement sensor, and its actuation curve is shown in FIG. 2. During the charging, the carbon nanotube yarn serving as the working electrode is gradually contracting, the contractile stroke of the carbon nanotube yarn reaches a maximum value of 16% when charging to 2.2 V; during the discharging, the contractile stroke of the carbon nanotube yarn is gradually reduced to 0. FIG. 3 shows the contractile stroke of the carbon nanotube yarn after applying a square wave voltage of 2.2 V. When the voltage was applied, the carbon nanotube yarn contracted and the contractile stroke gradually increased and reached 9% after 4 seconds, then the contraction state of the carbon nanotube yarn is maintained when the application of the voltage is removed, and the contractile stroke of the carbon nanotube yarn still does not decay after the applied voltage is removed for 450 s.

FIG. 4 shows a galvanostatic charge/discharge curve of a two-electrode aluminum ion battery system under different current densities in the embodiment of the present application. The capacity of the two-electrode aluminum ion battery system is 44 mAh·g⁻¹ under the current density of 200 mA g⁻¹, and the capacity of the two-electrode aluminum ion battery system is 38.2 mAh·g⁻¹ under the current density of 2000 mA g⁻¹. When the current density increases by 10 times, the capacity decays by only 13%, which shows that the two-electrode aluminum ion battery system provided in the embodiment of the present application has good magnification performance, and the coulomb efficiency is maintained at nearly 100% under different current densities.

The embodiment of the present application provides an actuation mechanism for an electrochemical artificial muscle system based on a battery system. The actuation mechanism mainly relies on the insertion and de-insertion of ions. During the charging, ions are inserted into an electrode material so that the electrode material expands and macroscopically exhibits contraction. During the discharging, ions are de-inserted from the electrode, the volume of the electrode is restored, and macroscopically exhibits that the length of the artificial muscle is restored. For the exact battery system, such as an aluminum ion battery system, during the charging, AlCl₄⁻ is embedded into a carbon nanotube fiber electrode so that the diameter of the carbon nanotube yarn is enlarged, thus realizing the length shrinking of the carbon nanotube yarn; during the discharging, AlCl₄⁻ detaches from the carbon nanotube yarn so that the radial size of the carbon nanotube yarn is restored and a fact that the length of the carbon nanotube yarn is restored is macroscopically exhibited.

### Example 2

The structure and fabricating process of an electrochemical artificial muscle testing device in this example are the same as those in example 1, and the same parts are not illustrated herein. The difference between example 2 and example 1 mainly lies in that:

### 1) Preparation of Artificial Muscle Yarns

one end of a graphene yarn vertically hung on a twisting motor, and the other end of the graphene yarn hung with a certain weight (the mass of the weight was 1-10 g), to twist the graphene fiber at the rotation speed of 0-200 rpm until a helical structure was formed;

### 2) Construction of Testing System

Anhydrous aluminum chloride was added into [EMIm]Cl to obtain an EMImAlCl₄ ionic liquid, and the EMImAlCl₄ ionic liquid was used as an electrolyte; the graphene fiber and the aluminum foil were each independently in contact with the electrolyte by using the EMImAlCl₄ ionic liquid as an electrolyte, the twisted graphene fiber as a working electrode and an aluminum foil as a counter electrode, to construct a two-electrode aluminum ion battery system.

### Example 3

The structure and fabricating process of an electrochemical artificial muscle testing device in this example are the same as those in example 1, and the same parts are not illustrated herein. The difference between example 3 and example 1 mainly lies in that:

1) Preparation of Artificial Muscle Fibers one end of a graphite fiber vertically hung on a twisting motor, and the other end of the graphite fiber hung with a certain weight (the mass of the weight was 1-10 g), to twist the graphite fiber at the rotation speed of 0-200 rpm until a helical structure was formed.

### 2) Construction of Testing System

Anhydrous aluminum chloride was added into [EMIm]Cl to obtain an EMImAlCl₄ ionic liquid, and the EMImAlCl₄ ionic liquid as an electrolyte; the graphite fiber and the aluminum foil were each independently in contact with the electrolyte by using the twisted graphite fiber as a working electrode and an aluminum foil as a counter electrode to construct a two-electrode aluminum ion battery system.

### Example 4

The structure and fabricating process of an electrochemical artificial muscle testing device in this example are the same as those in example 1, and the same parts are not illustrated herein. The difference between example 4 and example 1 mainly lies in that:

### 1) Preparation of Artificial Muscle Fibers

one end of a carbon fiber vertically hung on a twisting motor, and the other end of the carbon fiber hung with a certain weight (the mass of the weight was 1-10 g), to twist the carbon fiber at the rotation speed of 0-200 rpm until a helical structure was formed;

### 2) Construction of Testing System

Anhydrous aluminum chloride was added into [EMIm]Cl to obtain EMImAlCl₄ ionic liquid, and the EMImAlCl₄ ionic liquid was used as an electrolyte; the carbon fiber and the aluminum foil were each independently in contact with the electrolyte by using the twisted carbon fiber as a working electrode and an aluminum foil as a counter electrode, to construct a two-electrode aluminum ion battery system.

### Example 5

The structure and fabricating process of an electrochemical artificial muscle testing device in this example are the same as those in example 1, and the same parts are not illustrated herein. The difference between example 5 and example 1 mainly lies in that:

### 1) Preparation of Artificial Muscle Fibers

one end of a carbon nanotube fiber vertically hung on a twisting motor, and the other end of the carbon nanotube fiber hung with a certain weight (the mass of the weight was 1-10 g), to twist the carbon nanotube fiber at the rotation speed of 0-200 rpm until a helical structure was formed;

### 2) Construction of Testing System

Anhydrous aluminum chloride was added into urea to obtain an ionic liquid, and the ionic liquid was used as an electrolyte; the carbon nanotube fiber and the aluminum foil were each independently in contact with the electrolyte by using the twisted carbon nanotube fiber as a working electrode and an aluminum foil as a counter electrode, to construct a two-electrode aluminum ion battery system.

### Example 6

The structure and fabricating process of an electrochemical artificial muscle testing device in this example are the same as those in example 1, and the same parts are not illustrated herein. The difference between example 6 and example 1 mainly lies in that:

### 1) Preparation of Artificial Muscle Fibers

one end of a carbon nanotube yarn vertically hung on a twisting motor, and the other end of the carbon nanotube yarn hung with a certain weight (the mass of the weight was 1-10 g), to twist the carbon nanotube yarn at the rotation speed of 0-200 rpm until a helical structure was formed;

### 2) Construction of Testing System

The carbon nanotube yarn and the aluminum foil were each independently in contact with the electrolyte by using an organic solution of lithium-ion as an electrolyte, the twisted carbon nanotube yarn as a working electrode, and an aluminum foil as a counter electrode, to construct a two-electrode aluminum ion battery system.

### Example 7

The structure and fabricating process of an electrochemical artificial muscle testing device in this example are the same as those in example 1, and the same parts are not illustrated herein. The difference between example 7 and example 1 mainly lies in that:

### 1) Preparation of Artificial Muscle Yarns

one end of a carbon nanotube yarn vertically hung on a twisting motor, and the other end of the carbon nanotube yarn hung with a certain weight (the mass of the weight was 1-10 g), to twist the carbon nanotube yarn at the rotation speed of 0-200 rpm until a helical structure was formed;

### 2) Construction of Testing System

The carbon nanotube yarn and the aluminum foil were each independently in contact with the electrolyte by using a zinc ion solution as an electrolyte, the twisted carbon nanotube yarn as a working electrode, and an aluminum foil as a counter electrode, to construct a two-electrode zinc ion battery system.

The applicant of this case tests the electrochemical artificial muscle system in examples 2-7 concerning the testing method in example 1. The testing results are consistent with those in example 1.

The artificial muscle system based on the aluminum ion battery system provided by the embodiment of the present application has shrinkage retention characteristic, i.e., artificial muscle yarns shrink when a voltage is applied, and after the voltage is removed, the contraction state of the artificial muscle yarn is remained, within almost no any decay within 450 s, thereby overcoming the drawback that the existing artificial muscle yarns need to keep stimulus to maintain the contraction of the artificial muscle.

The artificial muscle system based on the aluminum ion battery system provided by the embodiment of the present application couples a driving function with energy storage that can store externally input energy during the driving of the artificial muscle yarns, and the stored energy can power an electrical appliance, thereby avoiding the waste of the energy.

The artificial muscle system based on the aluminum ion battery system provided by the embodiment of the present application is simple in fabricating process, can be produced on a large scale, and is beneficial to productization.

It should be understood that the above examples are only for illustrating the technical thinking and features of the present application for the purpose that individuals familiar with this technology understand the content of the present application and implement it, and accordingly do not limit the scope of protection of the present application. Equivalent changes or modifications made according to the spirit of the present application will be included within the scope of the protection of the present application.

What is claimed is:

1. An electrochemical artificial muscle system, comprising:
a working electrode, wherein the working electrode comprises a conductive yarn having a coiled or helical structure;
a counter electrode, wherein the counter electrode comprises selected metal elements; and
an electrolyte, wherein the electrolyte comprises an ionic liquid containing selected ions, and the selected ions comprises the selected metal elements;
wherein the working electrode and the counter electrode are in contact with the electrolyte,
when a specified voltage is applied to the working and counter electrodes to charge the electrochemical artificial muscle system, the selected ions are allowed to be inserted into the conductive yarn, wherein the conductive yarn contracts along a self-length direction, and when the specified voltage applied to the working and counter electrodes is removed, the inserted ions are lock in the conductive yarn and make the conductive yarn keep a contraction state;
when the electrochemical artificial muscle system is externally discharged, the selected ions are allowed to detach from the conductive yarn, wherein the conductive yarn is restored to an initial length.

2. The electrochemical artificial muscle system according to claim 1, wherein the working electrode is obtained by twisting the conductive yarn until forming the coiled or helical structure.

3. The electrochemical artificial muscle system according to claim 1, wherein a twist of the conductive yarn having the coiled or helical structure is 1000 r/m-5000 r/m; and a diameter of the conductive yarn having the coiled or helical structure is 10 μm-500 μm.

4. The electrochemical artificial muscle system according to claim 1, wherein the conductive yarn comprises one or a combination of more than two of a carbon nanotube yarn, a graphene yarn, a graphite fiber, and a carbon fiber.

5. The electrochemical artificial muscle system according to claim 1, wherein the selected ions comprise one of AlCl₄⁻, a lithium-ion, and a zinc ion; and the ionic liquid comprises an EMImAlCl₄ ionic liquid or a salt solution containing lithium ions and/or zinc ions.

6. The electrochemical artificial muscle system according to claim 1, wherein the selected metal elements contained in the counter electrode comprise one or more than two of aluminum, lithium, and zinc elements; and a material of the counter electrode comprises an alloy formed by one or more than two metals selected from aluminum, lithium, and zinc.

7. The electrochemical artificial muscle system according to claim 1, wherein when the specified voltage applied to the working and counter electrodes is removed, a longest time when the conductive yarn keeps the contraction state is 450 s;
an electrochemical capacity of the working electrode is 20-100 mAh·g⁻¹; and
the specified voltage is 0-2.2 V.

8. An electrochemical artificial muscle testing device, comprising:
the electrochemical artificial muscle system according to claim 1, wherein a first end of the working electrode being is fixed on an electrode clamp;
a load fixedly connected to a second end of the working electrode and configured to pull the working electrode along a length direction of the working electrode; and
a displacement monitoring mechanism at least configured for measuring a displacement of the load.

9. The electrochemical artificial muscle testing device according to claim 8, wherein the displacement monitoring mechanism comprises a displacement sensor.

10. The electrochemical artificial muscle testing device according to claim 8, further comprising a cathode and an anode, wherein
the cathode is electrically connected to the working electrode,
the anode is electrically connected to the counter electrode, and
the cathode and the anode are configured for being connected to a power source and charging the electrochemical artificial muscle system, and for being electrically connected to the load and discharging the electrochemical artificial muscle system.

11. The electrochemical artificial muscle system according to claim 2, wherein a twist of the conductive yarn having the coiled or helical structure is 1000 r/m-5000 r/m; and a diameter of the conductive yarn having the coiled or helical structure is 10 μm-500 μm.

12. The electrochemical artificial muscle system according to claim 2, wherein the conductive yarn comprises one or a combination of more than two of a carbon nanotube yarn, a graphene yarn, a graphite fiber, and a carbon fiber.

13. The electrochemical artificial muscle testing device according to claim 8, wherein the working electrode is obtained by twisting the conductive yarn until forming the coiled or helical structure.

14. The electrochemical artificial muscle testing device according to claim 8, wherein a twist of the conductive yarn having the coiled or helical structure is 1000 r/m-5000 r/m; and a diameter of the conductive yarn having the coiled or helical structure is 10 μm-500 μm.

15. The electrochemical artificial muscle testing device according to claim 8, wherein the conductive yarn comprises one or a combination of more than two of a carbon nanotube yarn, a graphene yarn, a graphite fiber, and a carbon fiber.

16. The electrochemical artificial muscle testing device according to claim 8, wherein the selected ions comprise one of AlCl₄⁻, a lithium-ion, and a zinc ion; and the ionic liquid comprises an EMImAlCl₄ ionic liquid or a salt solution containing lithium ions and/or zinc ions.

17. The electrochemical artificial muscle testing device according to claim 8, wherein the selected metal elements contained in the counter electrode comprise one or more than two of aluminum, lithium, and zinc elements; and a material of the counter electrode comprises an alloy formed by one or more than two metals selected from aluminum, lithium, and zinc.

18. The electrochemical artificial muscle testing device according to claim 8, wherein when the specified voltage applied to the working and counter electrodes is removed, a longest time when the conductive yarn keeps the contraction state is 450 s;
an electrochemical capacity of the working electrode is 20-100 mAh·g⁻¹; and
the specified voltage is 0-2.2 V.
